(19) United States
(12) Patent Application Publication
(10) Pub. No.: US 2026/0063559 A1
(43) Pub. Date: Mar. 5, 2026

(54) AUTOMATIC FOOD INSPECTION DEVICE

(71) Applicant: AVer Information Inc., New Taipei City (TW)

(72) Inventor: Tzu-Cheng CHANG, New Taipei City (TW)

(21) Appl. No.: 19/001,384

(22) Filed: Dec. 24, 2024

(30) Foreign Application Priority Data
Aug. 30, 2024 (TW) 113132944

(51) Int. Cl. G01N 21/85; G01N 21/88; G01N 33/02; G01N 35/00

(52) U.S. Cl. CPC G01N 21/85; G01N 21/8851; G01N 2021/8592; G01N 33/02; G01N 35/00; G01N 2035/00465

(57) ABSTRACT

An automatic food inspection device includes an accommodation tank, a conveying track configured to convey an object to be tested, a vibrating motor, a first vibration reduction component, a second vibration reduction component, an image recognition module configured to determine the quality of the object to be tested, a photoelectric detecting module and a sorting module. The conveying track has first and second ends. The first and second vibration reduction components are respectively disposed at the first and second ends of the conveying track and are respectively configured to adjust a first amplitude and a second amplitude of the conveying track. The second amplitude is less than the first amplitude. The photoelectric detecting module is configured to detect the falling status of the object to be tested. The sorting module is configured to classify the object to be tested according to a determination result of the image recognition module.

### CROSS-REFERENCE TO RELATED APPLICATION

This application claims priority to Taiwan Application Serial Number 113132944, filed Aug. 30, 2024, which is herein incorporated by reference in its entirety.

### BACKGROUND

### Technical Field

The present disclosure relates to an automatic food inspection device.

### Description of Related Art

With the increasing progress of society, the quality of people life is also improving day by day. With the improvement of modern people's living conditions, tasting high-quality food has become an important part of life. Taking coffee as an example, coffee has become a very important beverage in modern people's daily lives. The unique coffee flavor depends on the selection of coffee beans and the roasting technology of the barista. Different temperatures and roasting times may bring different tastes to the coffee. The quality of the coffee is starting from the selection of coffee beans.

With the increasing advancement of science and technology, machines are used to screen and classify non-defective products and defective products of beans such as coffee beans, or grains, and are also widely used in large food factories or businesses. However, for non-intensive roasting users, there is a need to provide a device and a method that can steadily screen coffee beans. Therefore, such a demand exists in the manufacturers and the industry.

### SUMMARY

One technical aspect of the present disclosure is an automatic food inspection device.

According to one embodiment of the present disclosure, an automatic food inspection device includes an accommodation tank, a conveying track, a vibrating motor, a first vibration reduction component, a second vibration reduction component, an image recognition module, a photoelectric detecting module and a sorting module. The accommodation tank is configured to accommodate an object to be tested. The conveying track is configured to convey the object to be tested, and has a first end and a second end, the first end being adjacent to the accommodation tank, and the first end having a fallen object placement area for carrying the object to be tested. The vibrating motor is disposed around the fallen object placement area of the conveying track, and configured to drive the object to be tested to move from the first end to the second end of the conveying track. The first vibration reduction component is disposed at the first end of the conveying track, and configured to adjust a first vibration amplitude of the conveying track. The second vibration reduction component is disposed at the second end of the conveying track, and configured to adjust a second vibration amplitude of the conveying track, wherein the second vibration amplitude is less than the first vibration amplitude. The image recognition module is adjacent to the second end of the conveying track, and configured to determine the quality of the object to be tested. The photoelectric detecting module is adjacent to the image recognition module, and configured to detect the falling status of the object to be tested. The sorting module is adjacent to the image recognition module, and configured to classify the object to be tested according to a determination result of the image recognition module.

In one embodiment of the present disclosure, the fallen object placement area further includes a buffer area. The buffer area is located on a side of the fallen object placement area close to the second end of the conveying track, and configured such that the object to be tested is arranged and moves to the second end in sequence.

In one embodiment of the present disclosure, the vibrating motor is located below the buffer area and fixed to the conveying track.

In one embodiment of the present disclosure, an elastic force of the second vibration reduction component is greater than that of the first vibration reduction component.

In one embodiment of the present disclosure, a rotating shaft of the vibrating motor is perpendicular to a forward direction of the object to be tested.

In one embodiment of the present disclosure, the conveying track includes at least one U-shaped track.

In one embodiment of the present disclosure, the automatic food inspection device further includes a bean dispensing tank. The bean dispensing tank is located below the accommodation tank.

In one embodiment of the present disclosure, the width of the first end is greater than that of the second end.

In one embodiment of the present disclosure, the first vibration reduction component and the second vibration reduction component are connected to the conveying track respectively by a clamping component.

In one embodiment of the present disclosure, the first vibration reduction component and the second vibration reduction component include a screw, a gasket, a damping ring, and a spring.

In one embodiment of the present disclosure, wherein when the screw is pressed to the spring so that an elastic force is generated, the second vibration reduction component has a limit on the second vibration amplitude of the conveying track.

In one embodiment of the present disclosure, the first vibration reduction component and the second vibration reduction component include elastic elements integrally formed by rubber, silicone or flexible plastics.

In one embodiment of the present disclosure, the conveying track includes a first segment and a second segment, the first segment is wide to narrow in track width, and the track width of the second segment is less than that of the first segment.

In one embodiment of the present disclosure, the vibrating motor is disposed at the tail end of the first segment.

In one embodiment of the present disclosure, the vibrating motor includes a center-of-gravity block.

In one embodiment of the present disclosure, the image recognition module includes a first image sensor and a second image sensor configured to detect an image status of the fallen objects to be tested.

In one embodiment of the present disclosure, the image recognition module further includes a transparent track configured to carry the objects to be tested.

In one embodiment of the present disclosure, the transparent track and the conveying track are separately configured to form a gap.

In one embodiment of the present disclosure, the photoelectric detecting module includes multiple detectors configured to detect the falling status of the object to be tested.

In the above-mentioned embodiments of the present disclosure, since the automatic food inspection device includes the first vibration reduction component and the second vibration reduction component, the first vibration reduction component is disposed at the first end of the conveying track and configured to adjust the first vibration amplitude of the conveying track, the second vibration reduction component is disposed at the second end of the conveying track and configured to adjust the second vibration amplitude of the conveying track, where the second vibration amplitude is less than the first vibration amplitude, the vibrating motor can drive coffee beans to move forward to the second end by means of intense vibration at the first end of the conveying track, and the suppression of the vibration amplitude of the second end means that the coffee beans can fall in sequence more steadily and enter the image recognition module, the automatic food inspection device can supply beans steadily and screen the coffee beans, thereby improving the quality of life of consumers.

### BRIEF DESCRIPTION OF THE DRAWINGS

Aspects of the present disclosure may be best understood from subsequent embodiments when read in conjunction with the drawings. Note that, in accordance with standard practices in this industry, various features are not drawn to scale. In fact, the dimensions of the various features may be arbitrarily increased or decreased for clarity of argument.

FIG. 1 illustrates a perspective view of an automatic food inspection device according to one embodiment of the present disclosure;

FIG. 2 illustrates a top view of a feeding module of the automatic food inspection device in FIG. 1;

FIG. 3 illustrates a side view of the feeding module of the automatic food inspection device in FIG. 1;

FIG. 4 illustrates a front view of the feeding module of the automatic food inspection device in FIG. 1;

FIG. 5 illustrates a side view of a first vibration reduction component of the feeding module in FIG. 1;

FIG. 6 illustrates a schematic diagram showing partial components of an image recognition module in FIG. 1;

FIG. 7 illustrates a side view of a photoelectric detecting module and a sorting module in FIG. 1, and

FIG. 8 illustrates a flow chart of an object to be tested supply method of an automatic food inspection device according to one embodiment of the present disclosure.

### DETAILED DESCRIPTION

The following embodiments of the present disclosure provide a number of different embodiments, or examples, for implementing different characteristics of the subject matter provided. Specific examples of components and arrangements are described below to simplify the case. Obviously, these examples are examples only and are not intended as limitations. In addition, component symbols and/or letters may be repeated in each example of the case. Such repetition is intended for the purpose of simplicity and clarity, and does not itself specify the relationship between the various embodiments and/or configurations discussed.

Spatial relative terms such as “below”, “under”, “lower”, “above” and “upper” may be used for descriptive purposes herein to describe the relation of one element or feature to another as shown in the drawings. The spatial relative terms are intended to encompass different orientations of devices in use or operation other than those shown in the drawings. The devices may be oriented in other ways (to rotate 90 degrees or otherwise) and spatial relative descriptors used herein may be interpreted accordingly.

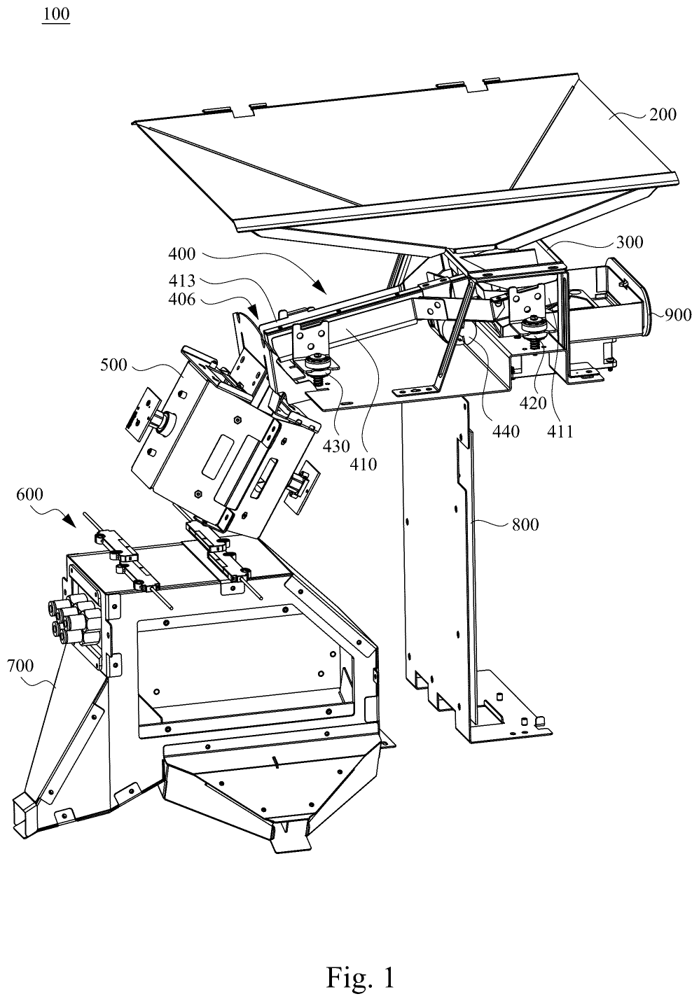

FIG. 1 shows a perspective view of an automatic food inspection device 100 according to one embodiment of the present disclosure. Referring to FIG. 1, an automatic food inspection device 100 includes an accommodation tank 200, a bean dispensing tank 300, a feeding module 400, an image recognition module 500, a photoelectric detecting module 600, a sorting module 700, an analysis control module 800 and a dust collection module 900. The accommodation tank 200 is configured to accommodate objects to be tested. The bean dispensing tank 300 is located below the accommodation tank 200 and configured to dispense the objects to be tested in the accommodation tank 200 to two tracks in the feeding module 400 (detailed below). The feeding module 400 is located below the bean dispensing tank 300 and configured to convey the objects to be tested so that the objects to be tested move forward one by one. The image recognition module 500 is adjacent to the feeding module 400 and configured to determine the quality of the objects to be tested. The photoelectric detecting module 600 is adjacent to the image recognition module 500 and configured to detect the falling status of the objects to be tested. The sorting module 700 is adjacent to the image recognition module 500 and configured to classify the objects to be tested according to a determination result of the image recognition module 500. The analysis control module 800 is configured to change a rotating speed of a vibrating motor 440 of the feeding module 400 according to information of the image recognition module 500 and the photoelectric detecting module 600. The dust collection module 900 is located below the feeding module 400 and configured to collect dust generated when the object to be tested falls. In some embodiments, the objects to be tested may be food, for example, beans such as coffee beans, or grains, but the present disclosure is not limited thereto.

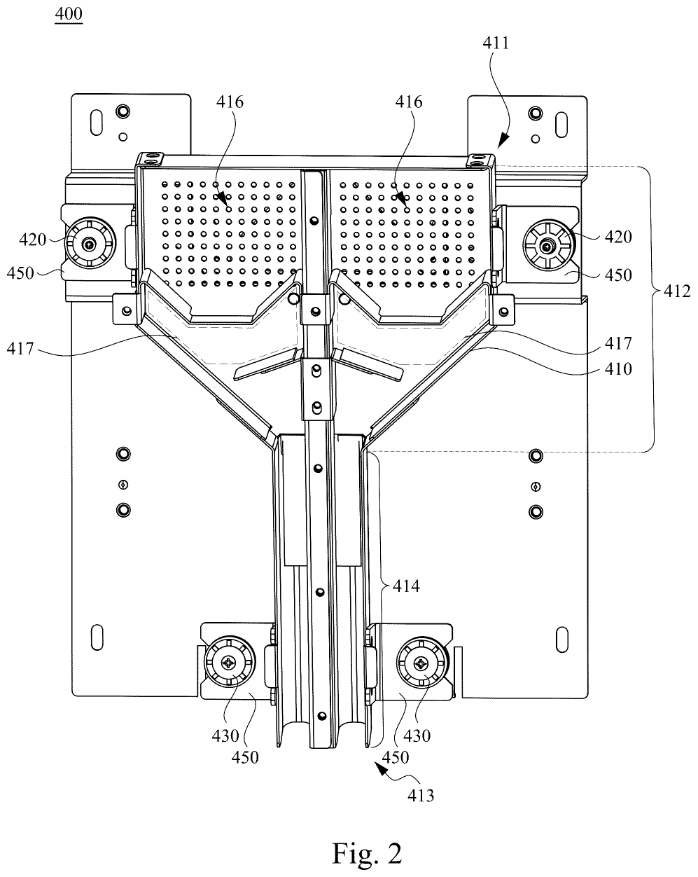

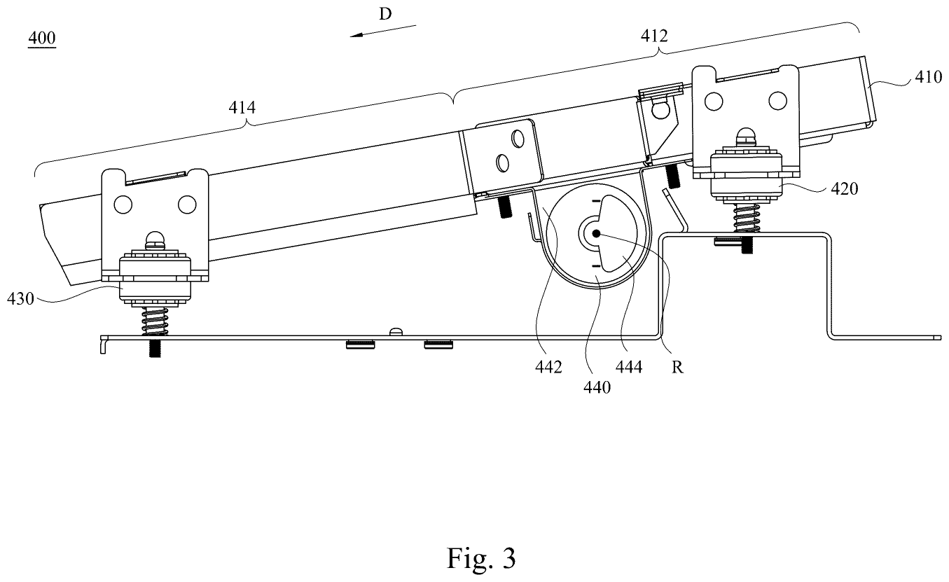

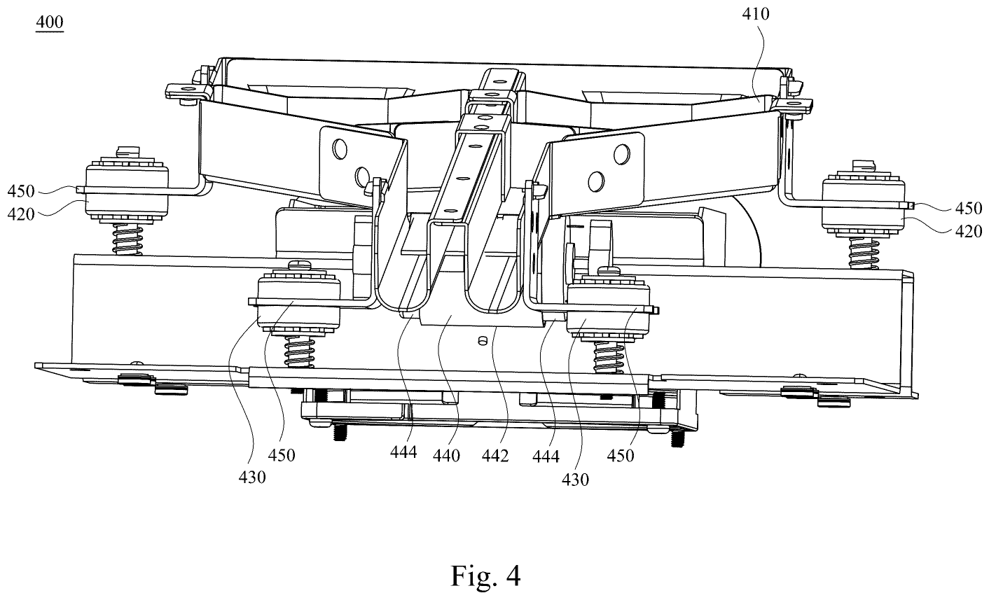

FIG. 2 illustrates a top view of the feeding module 400 of the automatic food inspection device 100 in FIG. 1. FIG. 3 illustrates a side view of the feeding module 400 of the automatic food inspection device 100 in FIG. 1. FIG. 4 illustrates a front view of the feeding module 400 of the automatic food inspection device 100 in FIG. 1. Referring to FIGS. 2-4, the feeding module 400 includes a conveying track 410, a first vibration reduction component 420, a second vibration reduction component 430, and the vibrating motor 440.

The conveying track 410 is configured to convey the objects to be tested and has a first end 411 and a second end 413, the first end 411 is adjacent to the accommodation tank 200, and the first end 411 has a fallen object placement area 416 for carrying the objects to be tested. In some embodiments, the fallen object placement area 416 further includes a buffer area 417 located on a side of the fallen object placement area 416 close to the second end 413 of the conveying track 410. In some embodiments, the conveying track 410 includes a first segment 412 and a second segment 414, the first segment 412 is wide to narrow in track width, and the track width of the second segment 414 is less than that of the first segment 412. That is, the width of the first end 411 is greater than that of the second end 413. Through the flow restriction of the first segment 412, a large number of test objects falling in the fallen object placement area 416 can be reduced in quantity in segments and enter the second segment 414 in sequence, and linearly move forward in the second segment 414 to the second end 413.

The vibrating motor 440 is disposed around the fallen object placement area 416 of the conveying track 410 and configured to drive the objects to be tested to move from the first end 411 to the second end 413 of the conveying track 410. Referring to FIG. 3, in some embodiments, the vibrating motor 440 is disposed at the tail end of the first segment 412 to produce a maximum vibration effect, for example, the vibrating motor 440 is located below the buffer area 417 and fixed to the conveying track 410. In particular, the vibrating motor 440 is fixed to the conveying track 410 through a metal sheet 442, and the rotating direction of the vibrating motor 440 is the same as the forward direction D of the objects to be tested. In particular, the vibrating motor 440 uses a center-of-gravity shift of a center-of-gravity block 444 (meaning that the center of gravity is not on a rotating shaft R) to cause vibration of the motor when rotating, so as to drive the objects to be tested in the conveying track 410 above to move forward. In some embodiments of the present disclosure, the rotating shaft R of the vibrating motor 440 is perpendicular to the forward direction D of the objects to be tested.

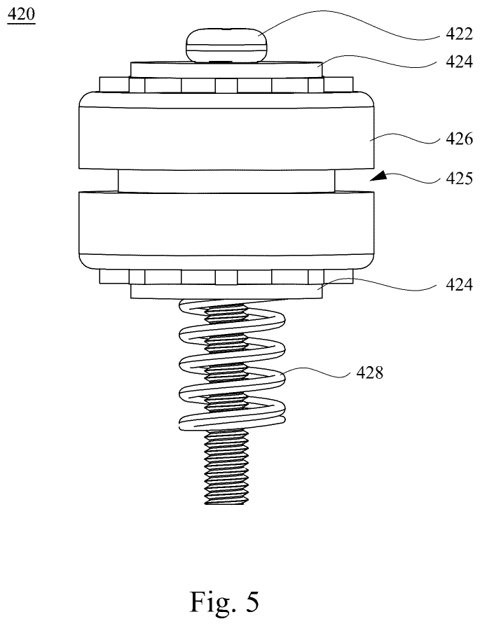

The first vibration reduction component 420 is disposed at the first end 411 of the conveying track 410 and configured to adjust a first vibration amplitude of the conveying track 410. The second vibration reduction component 430 is disposed at the second end 413 of the conveying track 410 and configured to adjust a second vibration amplitude of the conveying track 410, where the second vibration amplitude is less than the first vibration amplitude. For example, the first vibration reduction component 420 does not limit the vibration amplitude so that the objects to be tested stacked at the first segment 412 of the conveying track 410, after being separated from each other, enter the second segment 414 in sequence, thereby avoiding the objects to be tested being jammed at an inlet of the second segment 414; and the second vibration reduction component 430 limits the vibration amplitude of the second end 413 of the conveying track 410 to significantly reduce the jumping and tumbling of the objects to be tested and to improve the stability of the objects to be tested falling from the second end 413 of the conveying track 410, thereby avoiding misjudgments caused by the bouncing or tumbling problems of the objects to be tested when falling into the image recognition module 500. By a structural design in which the second vibration amplitude is less than the first vibration amplitude, the objects to be tested can steadily fall into the image recognition module 500 in sequence, and stable images can be obtained, so that the determination of the image recognition module 500 is more accurate, and the accuracy of inspection is improved. In one embodiment, the first vibration reduction component 420 and the second vibration reduction component 430 are connected to the conveying track 410 respectively by a clamping component 450. The clamping component 450 may be an L-shaped metal sheet, but the present disclosure is not limited thereto. FIG. 5 illustrates a side view of the first vibration reduction component 420 of the feeding module 400 in FIG. 1. Referring to FIG. 5, in some embodiments, the first vibration reduction component 420 may include a screw 422, a gasket 424, a damping ring 426, and a spring 428, but the present disclosure is not limited thereto. For example, the first vibration reduction component 420 may also be made by other means. In some embodiments, materials of the damping ring 426 include an elastic material such as rubber, silicone or flexible plastics. In other embodiments, the first vibration reduction component 420 and the second vibration reduction component 430 may be elastic elements integrally formed by rubber, silicone or flexible plastics, but the present disclosure is not limited thereto. In one embodiment, a groove 425 of the damping ring 426 is used to engage with the clamping component 450 (refer to FIG. 3). Furthermore, the second vibration reduction component 430 may also have a similar structure, but the present disclosure is not limited thereto. In one embodiment, when the screw 422 is screwed but not pressed to the spring 428, the vibration reduction component (e.g., the first vibration reduction component 420) will not have any limit on the vibration. However, when the screw 422 is screwed more tightly and pressed to the spring 428 so that an elastic force is generated, the vibration reduction component (e.g., the second vibration reduction component 430) will have a certain limit on the amplitude. In some embodiments of the present disclosure, an elastic force of the second vibration reduction component 430 is greater than that of the first vibration reduction component 420.

Referring to FIG. 4, in some embodiments, the conveying track 410 includes at least one U-shaped track. In the present embodiment, the conveying track 410 includes two U-shaped tracks, but the present disclosure is not limited thereto. For example, one, two, or more U-shaped tracks may be provided for the conveying and check of a plurality of objects to be tested individually or simultaneously, which is all not divorced from the spirit and scope of protection of the present disclosure.

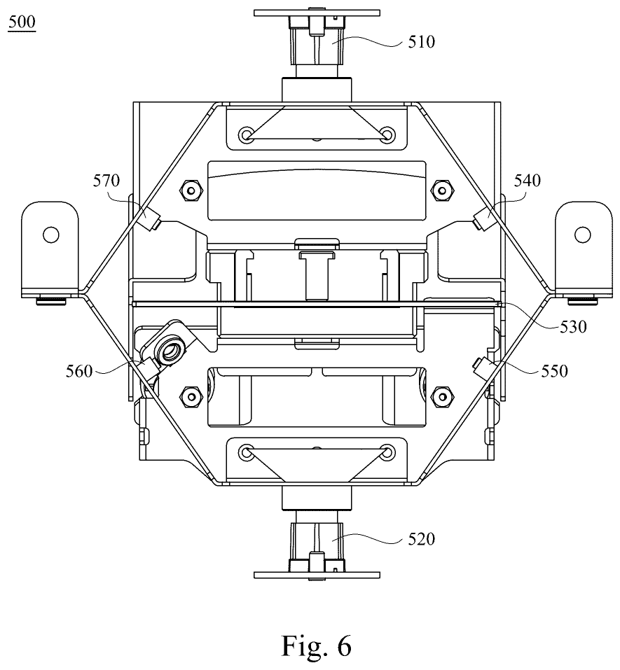

FIG. 6 illustrates a schematic diagram showing partial components of an image recognition module 500 in FIG. 1. Referring to FIG. 6, the objects to be tested, after sliding down from the second end 413 of the conveying track 410 of the feeding module 400, enters the image recognition module 500. The image recognition module 500 includes a first image sensor 510 and a second image sensor 520 which are configured to detect an image status of the fallen objects to be tested. The image status can be parameters such as profile, size, and color of the objects to be tested. The image recognition module 500 further includes a transparent track 530 configured to carry the objects to be tested. The first image sensor 510 and the second image sensor 520 are preferably disposed opposite to each other on both sides of the transparent track 530. Furthermore, the image recognition module 500 further includes a plurality of lighting devices such as a first lighting device 540, a second lighting device 550, a third lighting device 560 and a fourth lighting device 570 which are disposed on both sides of the transparent track 530 to illuminate the objects to be tested on the transparent track 530.

Referring also to FIGS. 6 and 1, the transparent track 530 and the feeding module 400 are separately configured for further conveying the objects to be tested, and one gap 406 is at least formed by means of separation due to the transparent track 530 and the feeding module 400. Therefore, the vibration of the feeding module 400 will not affect the transparent track 530, and also will not affect the first image sensor 510, the second image sensor 520, the first lighting device 540, the second lighting device 550, the third lighting device 560, and the fourth lighting device 570, thereby improving the accuracy and safety of image acquisition and further improving the service life of the automatic food inspection device 100.

In some embodiments, the first image sensor 510 and/or the second image sensor 520 may be, for example, a charge coupled device (CCD) or complementary metal-oxide semiconductor (CMOS). However, the present disclosure is not limited thereto. The first image sensor 510 and the second image sensor 520 are respectively a device that can convert optical signals of the objects to be tested into analog electrical signals, and then the analog electrical signals, after being processed by means of analog-to-digital conversion and color adjustment and the like, become digitalized image information of the objects to be tested, which is all not divorced from the spirit and scope of protection of the present disclosure.

The above digitalized image information of the objects to be tested can be transmitted to the analysis control module 800, and the analysis control module 800 determines the quality of the object to be tested according to image data of the image sensors. In some embodiments, the analysis control module 800 may receive or pre-load classification information about the quality of the object to be tested to capture images of front and back surfaces of the object to be tested using the first image sensor 510 and/or the second image sensor 520 when the object to be tested passes through the transparent track 530, and transmit the images to the analysis control module 800 for determination. However, the present disclosure is not limited thereto. In some embodiments, the analysis control module 800 can use artificial intelligence (AI) to learn, solve and identify the quality of the objects to be tested. In some embodiments, the analysis control module 800 can be connected to a network to make use of big data for analysis. In some embodiments, the analysis control module 800 can further be set up in the cloud or at a server, and the automatic food inspection device 100 can be connected to a network, transmit, through the network, an image of the object to be tested captured by the image recognition module 500 to the analysis control module 800 for determination, and transmit a determination result back to the automatic food inspection device 100 for use in classification of subsequent objects to be tested.

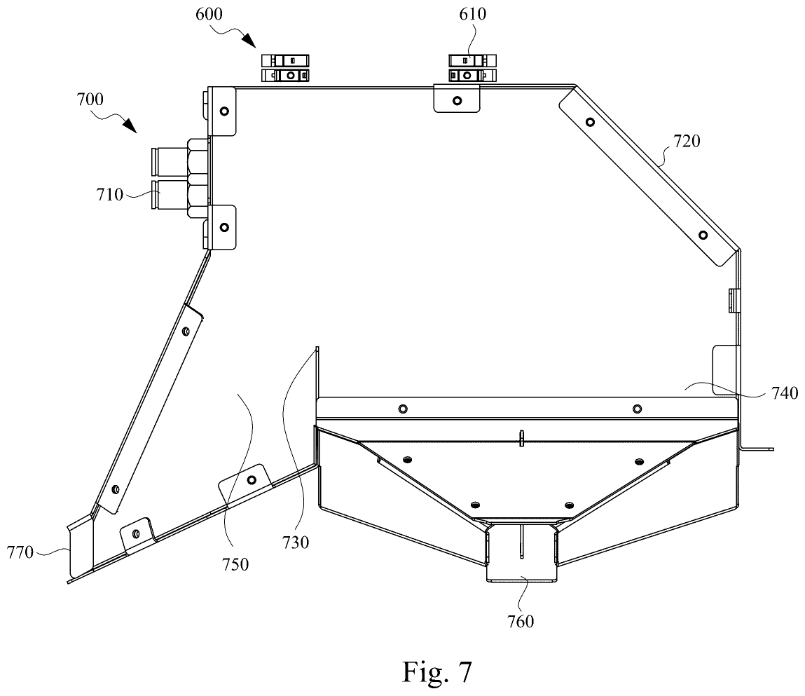

FIG. 7 illustrates a side view of the photoelectric detecting module 600 and the sorting module 700 in FIG. 1. Referring to FIG. 7, the photoelectric detecting module 600 includes a plurality of detectors 610 configured to detect the falling status of the object to be tested. The falling status of the object to be tested includes parameters such as a falling speed, the number of fallen objects to be tested in a fixed time. The object to be tested then enters a classification box 720 of the sorting module 700. At this point, the analysis control module 800 decides which side of the storage box does the object to be tested will be classified to, based on the quality of the object to be tested just determined. The sorting module 700 includes a plurality of high-pressure nozzles 710, the classification box 720, a separator 730, a first storage box 740, and a second storage box 750. The analysis control module 800 controls the high-pressure nozzles 710. When the object to be tested entering is determined to be of good quality, the high-pressure nozzles 710 will not operate, and the object to be tested will fall into the second storage box 750 along a falling trajectory, and finally leave from a second outlet 770. When the test object entering is determined to be of poor quality, the high-pressure nozzles 710 blow air to change the falling trajectory of the object to be tested, so that the object to be tested moves in the direction of the first storage box 740, and finally leaves from a first outlet 760.

In the following description, an object to be tested supply method of an automatic food inspection device is described.

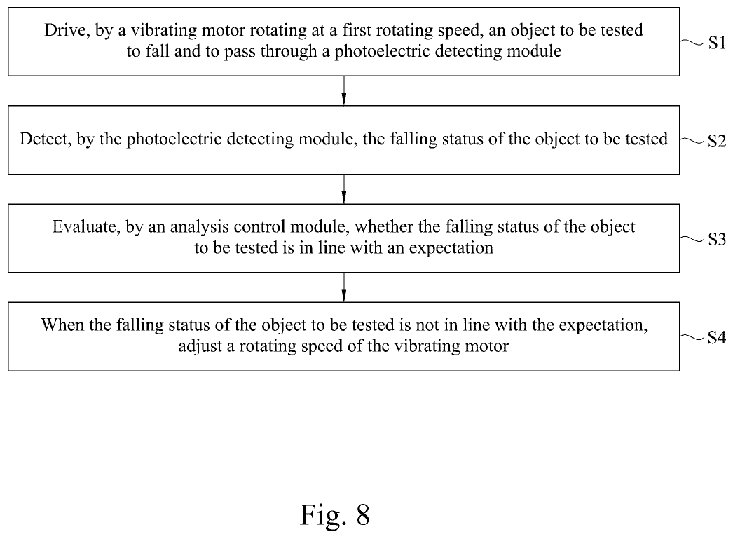

FIG. 8 illustrates a flow chart of an object to be tested supply method of an automatic food inspection device according to one embodiment of the present disclosure. Referring to FIG. 8, an object to be tested supply method of an automatic food inspection device includes the following steps: first, at step S1, a vibrating motor rotating at a first rotating speed drives an object to be tested to fall and to pass through a photoelectric detecting module. At step S2, the photoelectric detecting module is then used to detect the falling status of the object to be tested. Next, at step S3, the analysis control module is used to evaluate whether the falling status of the object to be tested is in line with an expectation. And finally, at step S4, when the falling status of the object to be tested is not in line with the expectation, a rotating speed of the vibrating motor is adjusted.

In some embodiments, the object to be tested supply method of an automatic food inspection device is not limited to steps S1-S4. For example, each of steps S1-S4 may include other more detailed steps. In some embodiments, steps S1-S4 may further include additional steps in between adjacent two steps, additional steps before step S1, and additional steps after step S4. The above steps will at least be described in detail in the following description.

Referring to FIG. 1, at the beginning of supplying the objects to be tested, the objects to be tested will, from the accommodation tank 200, pass through the bean dispensing tank 300 and then fall into the first end 411 of the conveying track 410 of the feeding module 400. At this time, the vibrating motor 440 rotates at the first speed to move forward the objects to be tested to the second end 413 of the conveying track 410 by means of vibrating, and finally, the objects to be tested enter the image recognition module 500. The objects to be tested, after leaving from the image recognition module 500, pass through the photoelectric detecting module 600. Subsequently, the photoelectric detecting module 600 transmits the number of the fallen objects to be tested in a certain period of time back to the analysis control module 800. The analysis control module 800 evaluates whether the number of the fallen objects to be tested in the certain period of time satisfies a preset value, and then, when the number of the fallen objects to be tested in the certain period of time does not satisfy the preset value, the analysis control module 800 issues a command to adjust the rotating speed of the vibrating motor 440. The command includes increasing the rotating speed of the vibrating motor 440 by the analysis control module 800 when the number of the fallen objects to be tested is less than a preset value range, and reducing the rotating speed of the vibrating motor 440 by the analysis control module 800 when the number of the fallen objects to be tested is greater than the present value range. In this way, when the objects to be tested that are different in size are detected, the objects to be tested can all fall at a stable speed to maintain good determination quality.

To sum up, since the automatic food inspection device includes the first vibration reduction component and the second vibration reduction component, the first vibration reduction component is disposed at the first end of the conveying track and configured to adjust the first vibration amplitude of the conveying track, the second vibration reduction component is disposed at the second end of the conveying track and configured to adjust a second vibration amplitude of the conveying track, where the second vibration amplitude is less than the first vibration amplitude, the vibrating motor can drive the objects to be tested to move forward to the second end by means of intense vibration at the first end of the conveying track, and the suppression of the vibration amplitude of the second end means that the objects to be tested can fall more steadily in sequence and enter the image recognition module, the automatic food inspection device can supply beans steadily and screen the objects to be tested (e.g., coffee beans), thereby improving the quality of life of consumers.

The foregoing outlines the features of several embodiments so that those skilled in the art may better understand the aspects of the present disclosure. Those skilled in the art should understand that they can easily use the present disclosure as a basis for designing or modifying other processes and structures to achieve the same purposes and/or to achieve the same advantages as the embodiments described herein. Those skilled in the art should also be aware that such equivalent constructions are not divorced from the spirit and scope of the present disclosure, and that, without deviating from the spirit and scope of the present disclosure; they may be subject here to various alterations, substitutions and alterations.

What is claimed is:

1. An automatic food inspection device, comprising:
an accommodation tank, configured to accommodate an object to be tested;
a conveying track, configured to convey the object to be tested, and having a first end and a second end, the first end being adjacent to the accommodation tank, and the first end having a fallen object placement area for carrying the object to be tested;
a vibrating motor, disposed around the fallen object placement area of the conveying track, and configured to drive the object to be tested to move from the first end to the second end of the conveying track;
a first vibration reduction component, disposed at the first end of the conveying track, and configured to adjust a first vibration amplitude of the conveying track;
a second vibration reduction component, disposed at the second end of the conveying track, and configured to adjust a second vibration amplitude of the conveying track, wherein the second vibration amplitude is less than the first vibration amplitude;
an image recognition module, disposed adjacent to the second end of the conveying track, and configured to determine the quality of the object to be tested;
a photoelectric detecting module, disposed adjacent to the image recognition module, and configured to detect a falling status of the object to be tested; and
a sorting module, disposed adjacent to the image recognition module, and configured to classify the object to be tested according to a determination result of the image recognition module.

2. The automatic food inspection device according to claim 1, wherein the fallen object placement area further comprises:
a buffer area, located on a side of the fallen object placement area close to the second end of the conveying track, and configured such that the object to be tested is arranged and moves to the second end in sequence.

3. The automatic food inspection device according to claim 2, wherein the vibrating motor is located below the buffer area and fixed to the conveying track.

4. The automatic food inspection device according to claim 1, wherein an elastic force of the second vibration reduction component is greater than that of the first vibration reduction component.

5. The automatic food inspection device according to claim 1, wherein a rotating shaft of the vibrating motor is perpendicular to a forward direction of the object to be tested.

6. The automatic food inspection device according to claim 1, wherein the conveying track comprises at least one U-shaped track.

7. The automatic food inspection device according to claim 1, further comprising:
a bean dispensing tank, located below the accommodation tank.

8. The automatic food inspection device according to claim 1, wherein the width of the first end is greater than that of the second end.

9. The automatic food inspection device according to claim 1, wherein the first vibration reduction component and the second vibration reduction component are connected to the conveying track respectively by a clamping component.

10. The automatic food inspection device according to claim 1, wherein the first vibration reduction component and the second vibration reduction component comprise a screw, a gasket, a damping ring, and a spring.

11. The automatic food inspection device according to claim 10, wherein when the screw is pressed to the spring so that an elastic force is generated, the second vibration reduction component has a limit on the second vibration amplitude of the conveying track.

12. The automatic food inspection device according to claim 10, wherein the first vibration reduction component and the second vibration reduction component comprise elastic elements integrally formed by rubber, silicone or flexible plastics.

13. The automatic food inspection device according to claim 1, wherein the conveying track includes a first segment and a second segment, the first segment is wide to narrow in track width, and the track width of the second segment is less than that of the first segment.

14. The automatic food inspection device according to claim 13, wherein the vibrating motor is disposed at the tail end of the first segment.

15. The automatic food inspection device according to claim 1, wherein the vibrating motor comprises a center-of-gravity block.

16. The automatic food inspection device according to claim 1, wherein the image recognition module comprises a first image sensor and a second image sensor configured to detect an image status of the fallen objects to be tested.

17. The automatic food inspection device according to claim 1, wherein the image recognition module further comprises a transparent track configured to carry the objects to be tested.

18. The automatic food inspection device according to claim 17, wherein the transparent track and the conveying track are separately configured to form a gap.

19. The automatic food inspection device according to claim 1, wherein the photoelectric detecting module comprises a plurality of detectors configured to detect the falling status of the object to be tested.
